(19) United States
(12) Patent Application Publication
(10) Pub. No.: US 2005/0010170 A1
(43) Pub. Date: Jan. 13, 2005

(54) IMPLANTABLE MEDICAL DEVICE WITH BENEFICIAL AGENT CONCENTRATION GRADIENT

(76) Inventors: John F Shanley, Redwood City, CA (US); Theodore L Parker, Danville, CA (US); Kinama Park, West Lafayette, IN (US)

Correspondence Address: BURNS DOANE SWECKER & MATHIS L L P, POST OFFICE BOX 1404, ALEXANDRIA, VA, 22313-1404, US

(21) Appl. No.: 10/777,283

(22) Filed: Feb. 11, 2004

(51) Int. Cl. A61M 31/00; A61M 37/00

(52) U.S. Cl. 604/93.01; 604/500; 128/898

(57) ABSTRACT

Abstract of the Disclosure

The implantable medical devices are configured to release at least one therapeutic agent from a matrix affixed to the implantable body with a release profile which is programable to the agent and treatment. The matrix is formed such that the concentration of the therapeutic agent in the matrix varies as a gradient relative to a surface of the implantable body. The change in the concentration gradient of the agent in the matrix directly controls the rate of elution of the agent from the matrix. The therapeutic agent matrix can be disposed in the stent or on surfaces of the stent in various configurations, including within volumes defined by the stent, such as openings, holes, or concave surfaces, as a reservoir of agent, and alternatively as a coating on all or a portion of the surfaces of the stent structure.

### Detailed Description of the Invention

### Cross Reference to Related Applications

This application is a Continuation-in-Part of U.S. Patent Application Serial No. 10/402,893 filed on March 28, 2003, which is incorporated herein by reference in its entirety.

### Background of Invention

FIELD OF THE INVENTION

The invention relates to a therapeutic agent delivery device which has a concentration gradient of the therapeutic agent contained within a matrix to provide release kinetics which are specifically programable for the particular agent, administration period, and release rate desired.

BACKGROUND

Implantable medical devices are sometimes used for delivery of a therapeutic agent, such as a drug, to an organ or tissue in the body. It is hoped that these devices may deliver agents to a wide variety of bodily systems to provide a wide variety of treatments.

One implantable medical device which has been used for local delivery of therapeutic agents is the coronary stent. Coronary stents are typically introduced percutaneously, and transported transluminally until positioned at a desired location. These devices are then expanded either mechanically, such as by the expansion of a mandrel or balloon positioned inside the device, or expand themselves by releasing stored energy upon actuation within the body. Once expanded within the lumen, these devices, called stents, become encapsulated within the body tissue and remain a permanent implant.

Of the many problems that may be addressed through stent-based local delivery of therapeutic agents, one of the most important is restenosis. Restenosis is a major complication that can arise following vascular interventions such as angioplasty and the implantation of stents. Simply defined, restenosis is a wound healing process that reduces the vessel lumen diameter by extracellular matrix deposition, neointimal hyperplasia, and vascular smooth muscle cell proliferation, and which may ultimately result in renarrowing or even reocclusion of the lumen. Despite the introduction of improved surgical techniques, devices, and pharmaceutical agents, the overall restenosis rate is still reported in the range of 25% to 50% within six to twelve months after an angioplasty procedure. To treat this condition, additional revascularization procedures are frequently required, thereby increasing trauma and risk to the patient.

One of the techniques under development to address the problem of restenosis is the use of surface coatings of various therapeutic agents on stents. U.S. Pat. No. 5,716,981, for example, discloses a stent that is surface-coated with a composition comprising a polymer carrier and paclitaxel (a well-known compound that is commonly used in the treatment of cancerous tumors). Known surface coatings, however, can provide little actual control over the release kinetics of therapeutic agents. These coatings are generally very thin, typically 5 to 8 microns deep. The surface area of the stent, by comparison is very large, so that the entire volume of the therapeutic agent has a very short diffusion path to discharge into the surrounding tissue. The ability to shape the release profiles from such systems is severely limited.

Accordingly, it would be desirable to provide a therapeutic agent delivery device with the ability to program the release kinetics to the particular agent, administration period, and release rate desired.

### Summary of Invention

The present invention relates to implantable medical devices for programable delivery of a therapeutic agent, methods of forming implantable medical devices, and methods for delivering therapeutic agents from implantable medical devices.

In accordance with one aspect of the invention, an implantable medical device configured to release at least one therapeutic agent therefrom is provided, wherein the device includes an implantable body; and a matrix affixed to the implantable body. The matrix contains the at least one therapeutic agent therein, and the matrix is formed such that the concentration of the therapeutic agent in the matrix varies as a continuous gradient relative to a surface of the implantable body.

In accordance with another aspect of the invention, a method of forming an implantable medical device configured to release at least one therapeutic agent therefrom is provided. The therapeutic agent is disposed in a matrix affixed to the body of the implantable medical device, and the concentration of the at least one therapeutic agent in the matrix varies as a continuous gradient relative to a surface of the body of the implantable medical device. The method involves forming a first homogeneous solution comprising the at least one therapeutic agent mixed with a polymeric binder, applying the first homogeneous solution to the body of the implantable medical device, solidifying the first homogeneous solution, thereby forming a first portion of the matrix, forming a second homogeneous solution comprising the polymeric binder, applying the second homogeneous solution to the first portion of the matrix, thereby at least partially liquifying the first portion of the matrix, and solidifying the second homogeneous solution, thereby forming a second portion of the matrix, wherein the concentration of the at least one therapeutic agent in the matrix is different in the first and second portions of the matrix.

In accordance with an additional aspect of the invention, a method of forming an implantable medical device configured to release at least one therapeutic agent therefreom is provided. The therapeutic agent is disposed in a matrix affixed to a body of the implantable medical device, and a concentration of the at least one therapeutic agent in the matrix varies as a continuous gradient relative to a surface of the implantable medical device body. The method involves forming a homogeneous solution comprising a polymeric binder and a solvent, evaporating the solvent in the homogeneous solution, thereby forming a matrix, exposing the matrix to a solution comprising the therapeutic agent for a time sufficient to produce a partial diffusion of the therapeutic agent into the matrix such that the concentration of the therapeutic agent varies in the matrix, and affixing the matrix to the implantable medical device body.

In accordance with a further aspect of the invention, a method for treating a patient by local delivery of at least one therapeutic agent is provided. The method involves delivering an implantable medical device into the body of a patient, the implantable medical device having a matrix affixed to a body of the implantable medical device with concentration of the at least one therapeutic agent in the matrix varying as a continuous gradient relative to a surface of the body of the implantable medical device. The method further involves delivering the therapeutic agent at a release rate and over an administration period determined by the gradient of therapeutic agent in the matrix.

### Brief Description of Drawings

The invention will now be described in greater detail with reference to the preferred embodiments illustrated in the accompanying drawings, in which like elements bear like reference numerals, and wherein:

FIG. 1 is a perspective view of one example of a stent according to the present invention.

FIG. 2 is a side view of a portion of the stent of FIG. 1.

FIG. 3 is a side cross sectional view of an example of an opening in a stent showing a matrix with one therapeutic agent having a concentration gradient.

FIG. 4 is a graph of the therapeutic agent concentration gradient of FIG. 3.

FIG. 5 is a graph of the release kinetics of the stent of FIG. 3.

FIG. 6 is a side cross sectional view of another example of an opening in a stent a matrix with one therapeutic agent having a concentration gradient.

FIG. 7 is a graph of the therapeutic agent concentration gradient of FIG. 6.

FIG. 8 is a graph of the release kinetics of the stent of FIG. 6.

FIG. 9 is a side cross sectional view of an example of an opening in a stent showing a matrix with two therapeutic agents having concentration gradients.

FIG. 10 is a graph of the therapeutic agent concentration gradients of FIG. 9.

FIG. 11 is a graph of the release kinetics of the stent of FIG. 9.

### Detailed Description

The invention relates to a medical device or stent having a matrix containing a therapeutic agent therein such that the concentration of agent in the matrix varies as a function of the position relative to the matrix surfaces. The agent may be any therapeutic agent that provides a beneficial effect after the deployment of the medical device and release of the agent from the matrix into the tissue of a mammal.

First, the following terms, as used herein, shall have the following meanings:

The terms "drug" and "therapeutic agent" are used interchangeably to refer to any therapeutically active substance that is delivered to a living being to produce a desired, usually beneficial, effect.

The term "matrix" or "biocompatible matrix" are used interchangeably to refer to a medium or material that, upon implantation in a subject, does not elicit a detrimental response sufficient to result in the rejection of the matrix. The matrix typically does not provide any therapeutic responses itself, though the matrix may contain or surround a therapeutic agent, and/or modulate the release of the therapeutic agent into the body. A matrix is also a medium that may simply provide support, structural integrity or structural barriers. The matrix may be polymeric, non-polymeric, hydrophobic, hydrophilic, lipophilic, amphiphilic, and the like. The matrix may be bioresorbable or non-bioresorbable.

The term "bioresorbable" refers to a matrix, as defined herein, that can be broken down by either chemical or physical process, upon interaction with a physiological environment. The matrix can erode or dissolve. A bioresorbable matrix serves a temporary function in the body, such as drug delivery, and is then degraded or broken into components that are metabolizable or excretable, over a period of time from minutes to years, preferably less than one year, while maintaining any requisite structural integrity in that same time period.

The term openings includes both through openings and recesses.

The term pharmaceutically acceptable refers to the characteristic of being non-toxic to a host or patient and suitable for maintaining the stability of a therapeutic agent and allowing the delivery of the therapeutic agent to target cells or tissue.

The term "polymer" refers to molecules formed from the chemical union of two or more repeating units, called monomers. Accordingly, included within the term "polymer" may be, for example, dimers, trimers and oligomers. The polymer may be synthetic, naturallyor semisynthetic. In preferred form, the term "polymer" refers to molecules which typically have a Mw greater than about 3000 and preferably greater than about 10,000 and a Mw that is less than about 10 million, preferably less than about a million and more preferably less than about 200,000. Examples of polymers include but are not limited to, poly-α-hydroxy acid esters such as, polylactic acid (PLLA or DLPLA), polyglycolic acid, polylactic-co-glycolic acid (PLGA), polylactic acid-co-caprolactone; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide); polyvinyl pyrrolidone; polyorthoesters; polysaccharides and polysaccharide derivatives such as polyhyaluronic acid, poly (glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclodextrin sulfobutyl ethers; polypeptides and proteins, such as polylysine, polyglutamic acid, albumin; polyanhydrides; polyhydroxy alkonoates such as polyhydroxy valerate, polyhydroxy butyrate, and the like.

The term primarily with respect to directional delivery, refers to an amount greater than about 50% of the total amount of therapeutic agent provided to a blood vessel.

The term restenosis refers to the renarrowing of an artery following an angioplasty procedure which may include stenosis following stent implantation.

The term "liquified" is used herein to define a component which is put in a liquid state either by heating the component to a temperature higher than its melting point, or glass transition temperature, or by dissolving the component in a solvent. The typical liquified materials of the present invention will have a viscosity of less than about 10,000 centipoise, and preferably less about 1,000 centipoise, and more preferably less than about 100 centipoise.

The term "homogeneously disposed" refers to a mixture in which each of the components are uniformly dispersed within the matrix.

The term "heterogeneously disposed" refers to a mixture in which the components are not mixed uniformly into a matrix.

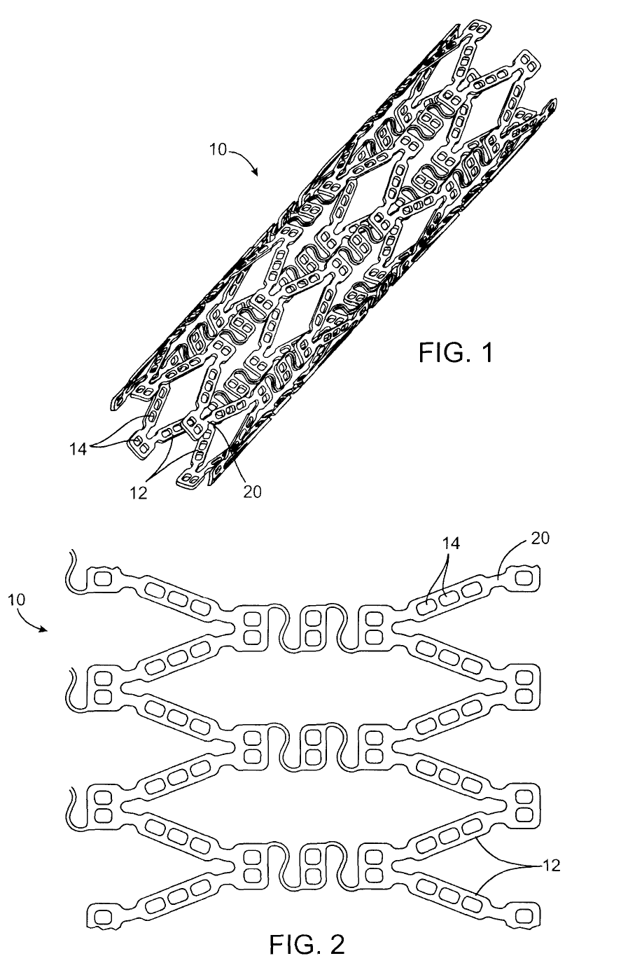

FIG. 1 illustrates one example of an implantable medical device in the form of a stent 10. Although the present invention will be described with reference to a stent, the invention can also be useful as other types of drug delivery implants including subcutaneous implants, embolization devices, implants for delivery of chemotherapeutic agents.

FIG. 2 is an enlarged flattened view of a portion of the stent of FIG. 1 illustrating one example of a stent structure including struts 12 interconnected by ductile hinges 20. The struts 12 include openings 14 which can be non-deforming openings containing the therapeutic agent and matrix. One example of a stent structure having non-deforming openings is shown in U.S. Patent No. 6,562,065 which is incorporated herein by reference in its entirety.

The implantable medical devices of the present invention are configured to release at least one therapeutic agent from a matrix affixed to the implantable body. The matrix is formed such that the concentration of the therapeutic agent in the matrix varies as a gradient relative to a surface of the matrix affixed to the implantable body. The dposition of a coating on a surface, such as by dipping or spraying may result in the phenomenon know as blooming by which the drug migrates to the surface resulting in increased concentration at the matrix surface. However, know coating methods do not achieve configurations in which a concentration in an area adjacent the matrix surface is less than a concentration of the drug at another part of the matrix. The present invention provides methods and devices by which an implantable medical device ca be designed to achieve a particular release profile by providing a concentration gradient of drug in a homogeneous polymer matrix in which the concentration gradient is provided other than by the phenomenon of blooming.

In one embodiment, the matrix is a polymeric material which acts as a binder or carrier to hold the agent in or on the stent and/or modulate the release of the agent from the stent. The polymeric material can be a bioresorbable or a non-bioresorbable material.

The therapeutic agent containing matrix can be disposed in the stent or on surfaces of the stent in various configurations, including within volumes defined by the stent, such as openings, holes, or concave surfaces, as a reservoir of agent, and alternatively as a coating on all or a portion of surfaces of the stent structure. When the therapeutic agent matrix is disposed within openings in the strut structure of the stent to form a reservoir, the openings may be partially or completely filled with matrix containing the therapeutic agent.

The concentration of agent in a local region of the matrix is the sum of the amount of agent dissolved in the matrix, in a so-called solid solution morphology, and the amount dispersed in that local region of the matrix, a so-called solid emulsion morphology. The relative amount of dissolved and dispersed agent in a region is controlled by the solubility of the agent in the matrix material. When the limit of solubility of the agent in the matrix material is reached, any additional agent will be in a dispersed second phase particulate morphology.

FIG. 3 is a cross section of the stent 10 and blood vessel 100 illustrating one example of an opening 14 arranged adjacent the vessel wall with a mural surface 26 abutting the vessel wall and a luminal surface 24 opposite the mural surface. The opening 14 of FIG. 3 contains a matrix 40 with a therapeutic agent illustrated by Os in the matrix. As can be seen in the example of FIG. 3, the concentration of the therapeutic agent (Os) is highest at the luminal side of the matrix 40 and lowest at the mural side of the matrix. The luminal side 24 of the stent 10 is also provided with a barrier layer 30. The barrier layer 30 causes the therapeutic agent to be delivered primarily to the mural side 26 of the stent.

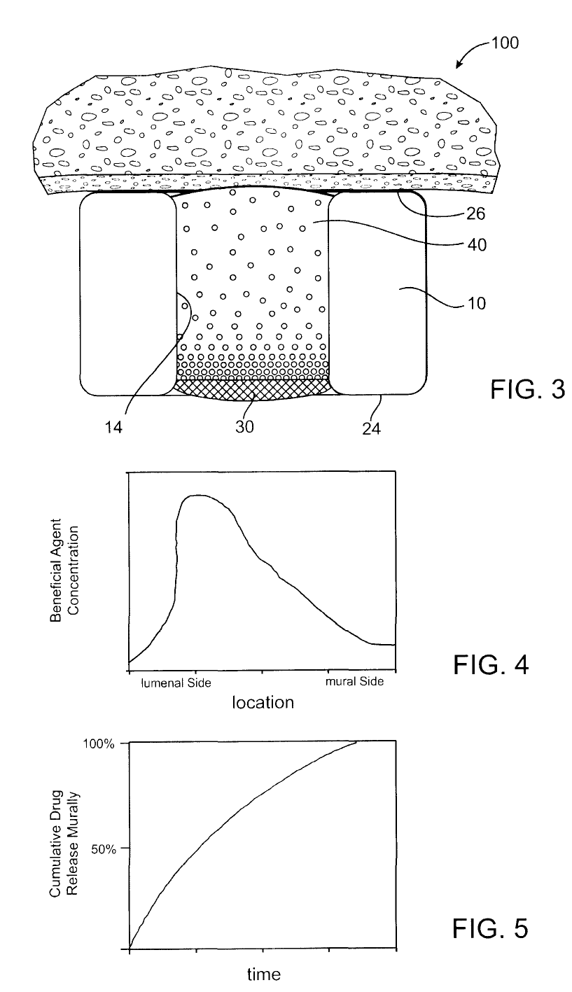

FIG. 4 illustrates graphically a concentration gradient similar to that depicted in FIG. 3 where the agent concentration in the matrix is highest in the middle of the stent or adjacent the luminally located barrier layer 30 and the agent concentration decreases moving toward the mural side of the matrix. The concentration gradient is described by the local concentration of the agent in matrix regions along a theoretical line substantially perpendicular to the surfaces of the matrix. A continuous agent concentration gradient is where the agent concentration in a volume of matrix varies in a blended fashion in moving between successive positions along the line substantially perpendicular to the matrix surface. Thus, if the matrix surface was substantially collinear with the stent surface and the matrix was sliced into a plurality of slices substantially parallel to the stent surface, the adjacent slices will have different agent concentrations. Alternately, the matrix surface may be contoured and the adjacent slices maybe similarly configured.

As illustrated in FIG. 3, the barrier layer 30 includes no therapeutic agent and the concentration gradient of therapeutic agent is provided in the matrix in the portion of the opening 14 not containing the barrier material. Alternatively, the barrier layer 30 may include some therapeutic agent and the concentration gradient may continue in part or all of the barrier layer.

As shown in FIG. 4, the change in agent concentration in the matrix is a continuous function of the position relative to the matrix surfaces. As shown in FIG. 5, the release kinetics of the system of FIGS. 3 and 4 can be essentially linear (essentially constant release rate over time) after an initial release. Such substantially linear release profiles are described in further detail in U.S. Patent Application Serial No. ________ (Attorney Docket No. 032304-107 ) filed on even date herewith which is incorporated herein by reference in its entirety.

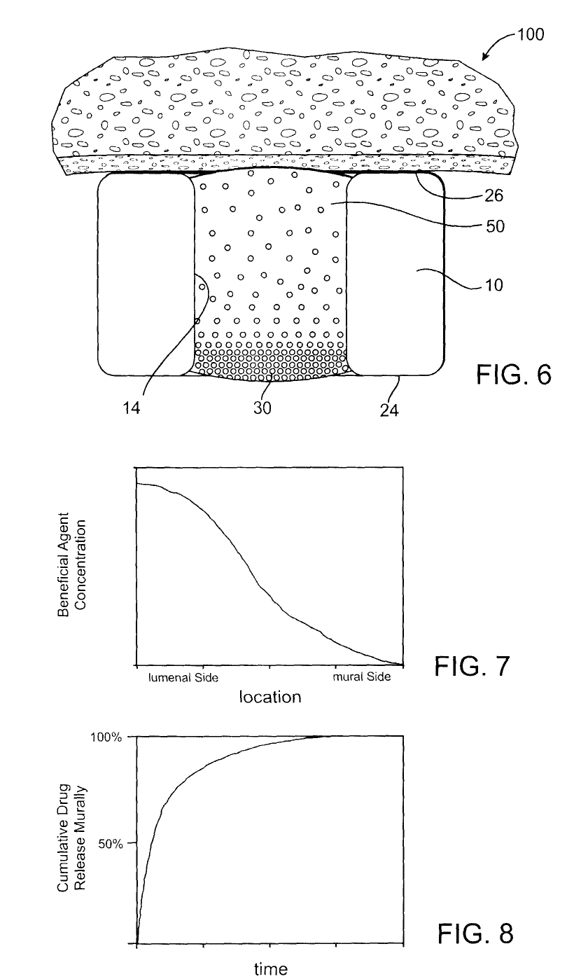

FIG. 6 illustrates a configuration of a matrix 50 in an opening 14 where the matrix and therapeutic agent concentration gradient are designed for rapid initial release of agent to the luminal side followed by a low level release for an extended time. The agent concentration in FIG. 6 is high at the luminal surface 24 of the matrix 50 and the concentration gradient will decrease steeply in the interior of the matrix. FIG. 7 illustrates the concentration gradient of the FIG. 6 example graphically. FIG. 8 illustrates the agent release over time for the example of FIGS. 6 and 7. Using careful specification of the agent concentration gradient in this example, substantially first order agent release kinetics with directionally controlled delivery may be obtained.

Since the matrix is created in a stepwise manner, as will be described below, individual chemical compositions and pharmacokinetic properties can be imparted to different areas of the matrix. Numerous useful arrangements of such matrix areas can be formed, some of which will be described herein. Each of the areas of the matrix may include one or more agents in the same or different proportions from one area to the next. The matrix may be solid, porous, or filled with other drugs or excipients. The agents may be homogeneously disposed or heterogeneously disposed in different areas of the matrix.

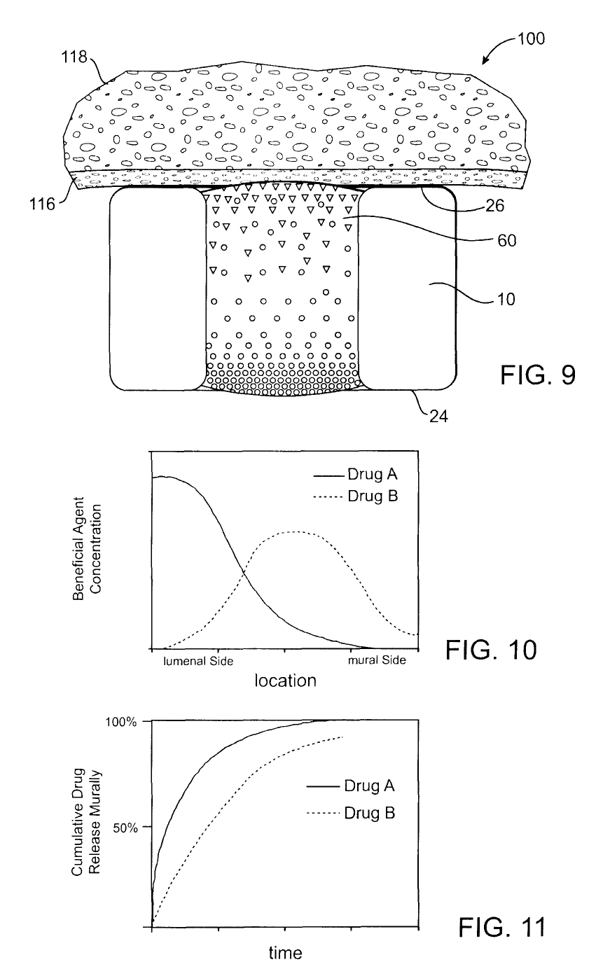

FIG. 9 illustrates an example of another stent 10 having a matrix 60 containing two agents with different concentration gradients. In FIG. 9, a first agent (Drug A) represented by Os is has a concentration gradient with a maximum concentration at a luminal side 24 of the stent. A second agent (Drug B) represented by ∇s has a concentration gradient with a maximum concentration at a mural side of the matrix. This configuration results in the delivery of two drugs in different primary delivery directions. For example, an antithrombotic agent (Drug A) may be delivered primarily luminally at a relatively quick initial release rate while an antirestenotic agent (Drug B) is delivered primarily murally with a different delivery profile having a more constant release rate and longer administration period. FIG. 10 illustrates graphically the agent concentration gradients of the first agent (Drug A) and the second agent (Drug B). FIG. 11 illustrates the cumulative release of the first and second agents (Drug A and Drug B) over time.

It is envisioned that the continuous agent concentration gradient will take a variety of forms depending on the desired administration period and rate of elution of the agent into the tissue surrounding the stent, as well as the desired direction of elution of agent from the stent, either mural or luminal. FIGS. 3-11 are merely illustrative of some of the concentration gradients which are useful. Further combinations of two or more agents with independent concentration gradients can provide a range of controlled release kinetic profiles of the agents from the matrix in or on the stent.

THERAPEUTIC AGENTS

Other therapeutic agents for use with the present invention may, for example, take the form of small molecules, peptides, lipoproteins, polypeptides, polynucleotides encoding polypeptides, lipids, protein-drugs, protein conjugate drugs, enzymes, oligonucleotides and their derivatives, ribozymes, other genetic material, cells, antisense oligonucleotides, monoclonal antibodies, platelets, prions, viruses, bacteria, eukaryotic cells such as endothelial cells, stem cells, ACE inhibitors, monocyte/macrophages and vascular smooth muscle cells. Such agents can be used alone or in various combinations with one another. For instance, anti-inflammatories may be used in combination with antiproliferatives to mitigate the reaction of tissue to the antiproliferative. The therapeutic agent may also be a pro-drug, which metabolizes into the desired drug when administered to a host. In addition, therapeutic agents may be pre-formulated as microcapsules, microspheres, microbubbles, liposomes, niosomes, emulsions, dispersions or the like before they are incorporated into the matrix. Therapeutic agents may also be radioactive isotopes or agents activated by some other form of energy such as light or ultrasonic energy, or by other circulating molecules that can be systemically administered.

Exemplary classes of therapeutic agents include antiproliferatives, antithrombins (i.e., thrombolytics), immunosuppressants, antilipid agents, anti-inflammatory agents, antineoplastics including antimetabolites, antiplatelets, angiogenic agents, anti-angiogenic agents, vitamins, antimitotics, metalloproteinase inhibitors, NO donors, nitric oxide release stimulators, anti-sclerosing agents, vasoactive agents, endothelial growth factors, beta blockers, AZ blockers, hormones, statins, insulin growth factors, antioxidants, membrane stabilizing agents, calcium antagonists (i.e., calcium channel antagonists), retinoids, anti-macrophage substances, antilymphocytes, cyclooxygenase inhibitors, immunomodulatory agents, angiotensin converting enzyme (ACE) inhibitors, anti-leukocytes, high-density lipoproteins (HDL) and derivatives, cell sensitizers to insulin, prostaglandins and derivatives, anti-TNF compounds, hypertension drugs, protein kinases, antisense oligonucleotides, cardio protectants, petidose inhibitors (increase blycolitic metabolism), endothelin receptor agonists, interleukin-6 antagonists, anti-restenotics, and other miscellaneous compounds.

Antiproliferatives include, without limitation, sirolimus, paclitaxel, actinomycin D, rapamycin, and cyclosporin.

Antithrombins include, without limitation, heparin, plasminogen, α₂-antiplasmin, streptokinase, bivalirudin, and tissue plasminogen activator (t-PA).

Immunosuppressants include, without limitation, cyclosporine, rapamycin and tacrolimus (FK-506), sirolumus, everolimus, etoposide, and mitoxantrone.

Antilipid agents include, without limitation, HMG CoA reductase inhibitors, nicotinic acid, probucol, and fibric acid derivatives (e.g., clofibrate, gemfibrozil, gemfibrozil, fenofibrate, ciprofibrate, and bezafibrate).

Anti-inflammatory agents include, without limitation, salicylic acid derivatives (e.g., aspirin, insulin, sodium salicylate, choline magnesium trisalicylate, salsalate, dflunisal, salicylsalicylic acid, sulfasalazine, and olsalazine), para-amino phenol derivatives (e.g., acetaminophen), indole and indene acetic acids (e.g., indomethacin, sulindac, and etodolac), heteroaryl acetic acids (e.g., tolmetin, diclofenac, and ketorolac), arylpropionic acids (e.g., ibuprofen, naproxen, flurbiprofen, ketoprofen, fenoprofen, and oxaprozin), anthranilic acids (e.g., mefenamic acid and meclofenamic acid), enolic acids (e.g., piroxicam, tenoxicam, phenylbutazone and oxyphenthatrazone), alkanones (e.g., nabumetone), glucocorticoids (e.g., dexamethaxone, prednisolone, and triamcinolone), pirfenidone, and tranilast.

Antineoplastics include, without limitation, nitrogen mustards (e.g., mechlorethamine, cyclophosphamide, ifosfamide, melphalan, and chlorambucil), methylnitrosoureas (e.g., streptozocin), 2-chloroethylnitrosoureas (e.g., carmustine, lomustine, semustine, and chlorozotocin), alkanesulfonic acids (e.g., busulfan), ethylenimines and methylmelamines (e.g., triethylenemelamine, thiotepa and altretamine), triazines (e.g., dacarbazine), folic acid analogs (e.g., methotrexate), pyrimidine analogs (5-fluorouracil, 5-fluorodeoxyuridine, 5-fluorodeoxyuridine monophosphate, cytosine arabinoside, 5-azacytidine, and 2',2'-difluorodeoxycytidine), purine analogs (e.g., mercaptopurine, thioguanine, azathioprine, adenosine, pentostatin, cladribine, and erythrohydroxynonyladenine), antimitotic drugs (e.g., vinblastine, vincristine, vindesine, vinorelbine, paclitaxel, docetaxel, epipodophyllotoxins, dactinomycin, daunorubicin, doxorubicin, idarubicin, epirubicin, mitoxantrone, bleomycins, plicamycin and mitomycin), phenoxodiol, etoposide, and platinum coordination complexes (e.g., cisplatin and carboplatin).

Antiplatelets include, without limitation, insulin, dipyridamole, tirofiban, eptifibatide, abciximab, and ticlopidine.

Angiogenic agents include, without limitation, phospholipids, ceramides, cerebrosides, neutral lipids, triglycerides, diglycerides, monoglycerides lecithin, sphingosides, angiotensin fragments, nicotine, pyruvate thiolesters, glycerol-pyruvate esters, dihydoxyacetone-pyruvate esters and monobutyrin.

Anti-angiogenic agents include, without limitation, endostatin, angiostatin, fumagillin and ovalicin.

Vitamins include, without limitation, water-soluble vitamins (e.g., thiamin, nicotinic acid, pyridoxine, and ascorbic acid) and fat-soluble vitamins (e.g., retinal, retinoic acid, retinaldehyde, phytonadione, menaqinone, menadione, and alpha tocopherol).

Antimitotics include, without limitation, vinblastine, vincristine, vindesine, vinorelbine, paclitaxel, docetaxel, epipodophyllotoxins, dactinomycin, daunorubicin, doxorubicin, idarubicin, epirubicin, mitoxantrone, bleomycins, plicamycin and mitomycin.

Metalloproteinase inhibitors include, without limitation, TIMP-1, TIMP-2, TIMP-3, and SmaPI.

NO donors include, without limitation, L-arginine, amyl nitrite, glyceryl trinitrate, sodium nitroprusside, molsidomine, diazeniumdiolates, S-nitrosothiols, and mesoionic oxatriazole derivatives.

NO release stimulators include, without limitation, adenosine.

Anti-sclerosing agents include, without limitation, collagenases and halofuginone.

Vasoactive agents include, without limitation, nitric oxide, adenosine, nitroglycerine, sodium nitroprusside, hydralazine, phentolamine, methoxamine, metaraminol, ephedrine, trapadil, dipyridamole, vasoactive intestinal polypeptides (VIP), arginine, and vasopressin.

Endothelial growth factors include, without limitation, VEGF (Vascular Endothelial Growth Factor) including VEGF-121 and VEG-165, FGF (Fibroblast Growth Factor) including FGF-1 and FGF-2, HGF (Hepatocyte Growth Factor), and Ang1 (Angiopoietin 1).

Beta blockers include, without limitation, propranolol, nadolol, timolol, pindolol, labetalol, metoprolol, atenolol, esmolol, and acebutolol.

Hormones include, without limitation, progestin, insulin, the estrogens and estradiols (e.g., estradiol, estradiol valerate, estradiol cypionate, ethinyl estradiol, mestranol, quinestrol, estrond, estrone sulfate, and equilin).

Statins include, without limitation, mevastatin, lovastatin, simvastatin, pravastatin, atorvastatin, and fluvastatin.

Insulin growth factors include, without limitation, IGF-1 and IGF-2.

Antioxidants include, without limitation, vitamin A, carotenoids and vitamin E.

Membrane stabilizing agents include, without limitation, certain beta blockers such as propranolol, acebutolol, labetalol, oxprenolol, pindolol and alprenolol.

Calcium antagonists include, without limitation, amlodipine, bepridil, diltiazem, felodipine, isradipine, nicardipine, nifedipine, nimodipine and verapamil.

Retinoids include, without limitation, all-trans-retinol, all-trans-14-hydroxyretroretinol, all-trans-retinaldehyde, all-trans-retinoic acid, all-trans-3,4-didehydroretinoic acid, 9-cis-retinoic acid, 11-cis-retinal, 13-cis-retinal, and 13-cis-retinoic acid.

Anti-macrophage substances include, without limitation, NO donors.

Anti-leukocytes include, without limitation, 2-CdA, IL-1 inhibitors, anti-CD116/CD18 monoclonal antibodies, monoclonal antibodies to VCAM, monoclonal antibodies to ICAM, and zinc protoporphyrin.

Cyclooxygenase inhibitors include, without limitation, Cox-1 inhibitors and Cox-2 inhibitors (e.g., CELEBREX®and VIOXX®).

Immunomodulatory agents include, without limitation, immunosuppressants (see above) and immunostimulants (e.g., levamisole, isoprinosine, Interferon alpha, and Interleukin-2).

ACE inhibitors include, without limitation, benazepril, captopril, enalapril, fosinopril sodium, lisinopril, quinapril, ramipril, and spirapril.

Cell sensitizers to insulin include, without limitation, glitazones, P par agonists and metformin.

Antisense oligonucleotides include, without limitation, resten-NG.

Cardio protectants include, without limitation, VIP, pituitary adenylate cyclase-activating peptide (PACAP), apoA-I milano, amlodipine, nicorandil, cilostaxone, and thienopyridine.

Petidose inhibitors include, without limitation, omnipatrilat.

Anti-restenotics include, without limitation, include vincristine, vinblastine, actinomycin, epothilone, paclitaxel, and paclitaxel derivatives (e.g., docetaxel).

Miscellaneous compounds include, without limitation, Adiponectin.

Agents may also be delivered using a gene therapy-based approach in combination with an expandable medical device. Gene therapy refers to the delivery of exogenous genes to a cell or tissue, thereby causing target cells to express the exogenous gene product. Genes are typically delivered by either mechanical or vector-mediated methods.

Some of the agents described herein may be combined with additives which preserve their activity. For example additives including surfactants, antacids, antioxidants, and detergents may be used to minimize denaturation and aggregation of a protein drug. Anionic, cationic, or nonionic detergents may be used. Examples of nonionic additives include but are not limited to sugars including sorbitol, sucrose, trehalose; dextrans including dextran, carboxy methyl (CM) dextran, diethylamino ethyl (DEAE) dextran; sugar derivatives including D-glucosaminic acid, and D-glucose diethyl mercaptal; synthetic polyethers including polyethylene glycol (PEG and PEO) and polyvinyl pyrrolidone (PVP); carboxylic acids including D-lactic acid, glycolic acid, and propionic acid; detergents with affinity for hydrophobic interfaces including n-dodecyl-β-D-maltoside, n-octyl-β-D-glucoside, PEO-fatty acid esters (e.g. stearate (myrj 59) or oleate), PEO-sorbitan-fatty acid esters (e.g. Tween 80, PEO-20 sorbitan monooleate), sorbitan-fatty acid esters (e.g. SPAN 60, sorbitan monostearate), PEO-glyceryl-fatty acid esters; glyceryl fatty acid esters (e.g. glyceryl monostearate), PEO-hydrocarbon-ethers (e.g. PEO-10 oleyl ether; triton X-100; and Lubrol. Examples of ionic detergents include but are not limited to fatty acid salts including calcium stearate, magnesium stearate, and zinc stearate; phospholipids including lecithin and phosphatidyl choline; CM-PEG; cholic acid; sodium dodecyl sulfate (SDS); docusate (AOT); and taumocholic acid.

MATRIX FORMATION METHODS

The agent matrix structure with the agent concentration gradient can be formed by several methods. According to one method, agent and polymer material are together converted into agent matrix reservoirs with an agent concentration gradient structure by first creating a homogeneous solution of agent and polymer carrier in a liquid form, such as in a solvent. One example of a solvent is one in which all agent and polymer are fully soluble at the respective concentrations desired for processing such that all ingredients are molecularly dissolved in the solvent.

Solvents may be water based, as when water soluble agents and water soluble polymers are the components of the agent delivery matrix. Alternatively, solvents can be mixtures of water with miscible organic solvents, such as dimethyl sulfoxide (DMSO), Nmethyl pyrrolidone (NMP), ethyl lactate (EL), dimethyl acetamide (DMAc), or simple alcohols. Additionally, non-aqueous solvents, predominantly organic solvents, can be suitable for non-water soluble polymers, such as poly(lactide-co-glycolide) polymers (PLGAs). Example organic solvents include DMSO, NMP, EL, anisole, chloroform, tetrahydrofuran (THF), xylene, and methylene chloride.

In the first method, steps (i) and (ii) are preformed followed by steps (iii) and (iv) which are repeated until the desired concentration gradient structure is obtained:

i) a solution comprised of suitable solvent and polymer material, and optionally a therapeutic agent, is introduced into an opening on the stent;

ii) the solvent is evaporated from the solution to form a first portion of matrix;

iii) a second solution is introduced which partially dissolves of otherwise liquifies the precedent material from step (ii) and allows partial mixing of the agent of precedent material and the components of the second solution to create a new hybrid solution in the cavity or hole in the stent; and

iv) the solvent is evaporated from the newly formed hybrid solution to provide a portion of matrix having a concentration gradient of the agent therein. By changing the composition of successive solutions there will result a final agent containing matrix where the agent is present in a continuously changing concentration relative to the depth of the matrix, termed a concentration gradient.

Although the process has been described employing a solvent, a similar process may use a solution without a solvent when the polymer is heated to achieve a liquefied or flowable condition.

Two general sequences of solution compositions can provide the concentration gradient structure of the invention. In a first sequence, one or several iterations of the same agent and polymer compositions are introduced as described followed by successive iterations of solutions containing polymer only. In this manner a first portion of matrix is fabricated with an agent containing solution followed by introduction of a second portion of matrix without agent. The second portion of matrix without the agent introduced just after the first portion containing agent will extract a portion of agent from the first portion into itself, creating a concentration gradient of the agent in the combined structure after the solvent has evaporated. Successive additions of solutions with polymer and no agent will only be able to dissolve the portion formed just before, which has successively smaller amounts of agent, so as the depth of the matrix is increased by successive additions the agent proportion will be successively decreasing, continuing the formation of an agent concentration gradient.

Although the first method has been described with reference to depositing in a hole or cavity, the matrix may also be formed on the stent or in the stent in other configurations including coatings or partial coatings in substantially the same manner. Coatings are generally less preferable than reservoirs, as the depth of reservoirs permits more complex morphologies.

In a second sequence, a first series of iterations are done with a solution containing matrix and an agent at a first agent concentration, followed by a second series of iterations done with a solution having matrix and the agent at a second agent concentration. The resultant matrix will have a agent concentration gradient where the absolute concentration is near the first concentration at one side of the matrix, at an intermediate concentration in the middle of the matrix, and near the second agent concentration at the opposite side of the matrix.

In a second method an agent concentration gradient is formed in the matrix by a process of diffusion. A matrix containing no agent is first prepared from solutions containing polymer. The formed matrix is then immersed in a solution containing an agent for a time period to allow a partial diffusion of the agent from the solution into the matrix, then the matrix is removed from the solution. The resultant matrix will have a relatively higher agent concentration near the surface(s) that contacted the solution and lower concentration toward the opposite side, thus forming an agent concentration gradient across the depth of the agent containing matrix.

This second method can be performed with a matrix in the form of a coating on a stent or a partial coating on a stent, with a matrix within openings in a stent, a matrix prior to placing the matrix on or in the stent, or another matrix configuration. When the matrix is formed within openings in a stent a barrier layer may be placed on one side of the opening to allow diffusion of the agent into the matrix from primarily one side of the opening. The barrier layer may subsequently be removed if delivery from the barrier side is desired. Additional barrier layers may be added after formation of the concentration gradient if desired. The barrier layer can be a bioresorbable or non-bioresorbable.

Example 1 - Formulation comprising a Gradient of a Therapeutic Agent

In the example below, the following abbreviations have the following meanings.

PLGA=poly(lactide-co-glycolide)

DMSO=Dimethyl sulfoxide

NMP= N-methylpyrrolidone

DMAC=Dimethyl acetamide

A first mixture of high molecular weight PLGA and a suitable organic solvent, such as DMSO, NMP, or DMAC 93% wt. is prepared. The mixture is loaded dropwise into openings in the stent, then the solvent is evaporated to begin formation of the barrier layer. A one or more additional barrier layers are laid over the first by the same method of filling polymer solution into the hole followed by solvent evaporation.

A second mixture of paclitaxel and low molecular weight PLGA, in a suitable organic solvent, such as DMSO, is introduced into openings in the stent over the barrier layer. The solvent is evaporated to form a drug filled therapeutic agent layer. The filling and evaporation procedure is repeated until the hole is filled to about 50% of its total volume with drug in therapeutic agent layer layered on top of the barrier layer.

Multiple layers of a third solution, of low molecular weight PLGA and a suitable organic solvent, such as DMSO, are then laid down over the therapeutic agent layer to form the concentration gradient. When each of the third solution layers is loaded into the stent, a portion of the layer beneath is incorporated in the new layer. In this way the matrix is formed containing a concentration gradient of paclitaxel agent.

Following implantation of the filled stent in vivo, the paclitaxel contained within the stent is delivered slowly over a time period of about 5 to about 60 days, preferably about 10 to about 30 days. The barrier layer prevents the therapeutic agent from being delivered out the barrier layer side of openings in the stent. The barrier layer completely degrades after the administration of the paclitaxel.

While the invention has been described in detail with reference to the preferred embodiments thereof, it will be apparent to one skilled in the art that various changes and modifications can be made and equivalents employed, without departing from the present invention.

1. An implantable medical device configured to release at least one therapeutic agent therefrom, the device comprising:
an implantable body; and
a matrix affixed to the implantable body, the matrix containing the at least one therapeutic agent therein, wherein the matrix is formed such that the concentration of the therapeutic agent in the matrix varies as a gradient relative to a surface of the matrix with a high concentration of the therapeutic agent spaced a distance from the matrix surface and a low concentration of the therapeutic agent at the matrix surface.

2. The device of Claim 1, wherein the matrix comprises a bioresorbable polymer.

3. The device of Claim 1, wherein the implantable body is substantially cylindrical.

4. The device of Claim 3, wherein the matrix is disposed in an opening in a mural surface of the implantable body.

5. The device of Claim 3, wherein the matrix is disposed in an opening in a luminal surface of the implantable body.

6. The device of Claim 3, wherein the concentration gradient of the therapeutic agent in the matrix increases from a minimum concentration of therapeutic agent at a luminal surface of the implantable body, reaches a maximum concentration of therapeutic agent in a center portion of the expandable body and then decreases towards a mural surface of the implantable body.

7. The device of Claim 3, wherein the concentration of the therapeutic agent in the matrix is higher at a mural surface of the implantable body than at a luminal surface of the implantable body.

8. The device of Claim 3, wherein the concentration of the therapeutic agent in the matrix is higher at a luminal surface of the implantable body than at a mural surface of the implantable body.

9. The device of Claim 1, wherein the matrix is disposed in an opening passing through the implantable body.

10. The device of Claim 1, wherein the matrix is disposed as a coating on the surface of the implantable body.

11. The device of Claim 1, wherein the therapeutic agent is dissolved in the matrix in a solid solution morphology.

12. The device of Claim 11, wherein the therapeutic agent is dispersed in the matrix in a solid emulsion morphology.

13. The device of Claim 1, wherein the therapeutic agent elutes from the matrix at a rate that is controlled by the concentration gradient of the therapeutic agent in the matrix.

14. The device of Claim 1, wherein the at least one therapeutic agent comprises a plurality of therapeutic agents.

15. The device of Claim 14, wherein the concentration of each of the therapeutic agents in the matrix vary with different continuous concentration gradients relative to the surface of the implantable body.

16. The device of Claim 1, wherein the therapeutic agent is selected from the group consisting of antithrombotic agents, antiproliferative agents, and antirestenotic agents.

17. The device of Claim 1, wherein the matrix is selected from the group consisting of poly(lactide-co-glycolide) (PLGA) and Poly vinylpyrrolidone (PVP).

18. The device of Claim 1, wherein the implantable body is substantially cylindrical, the matrix is disposed in a plurality of holes passing through the implantable body, and the matrix is separated from a luminal side of the implantable body by a barrier layer.

19. The device of Claim 18, wherein the therapeutic agent has a maximum concentration substantially adjacent to the barrier layer and a minimum concentration substantially adjacent to a mural surface of the implantable body.

20. The device of Claim 18, wherein the implantable body is substantially cylindrical and the holes are radial directed holes formed in a plurality of struts of the implantable body.

21. A method of forming an implantable medical device configured to release at least one therapeutic agent therefrom, wherein the therapeutic agent is disposed in a matrix affixed to the body of the implantable medical device, and wherein the concentration of the at least one therapeutic agent in the matrix varies as a continuous gradient relative to a surface of the body of the implantable medical device, the method comprising:
forming a first homogeneous solution comprising the at least one therapeutic agent mixed with a polymeric binder;
applying the first homogeneous solution to the body of the implantable medical device;
solidifying the first homogeneous solution, thereby forming a first portion of the matrix;
forming a second homogeneous solution comprising the polymeric binder;
applying the second homogeneous solution to the first portion of the matrix, thereby at least partially liquifying the first portion of the matrix; and
solidifying the second homogeneous solution, thereby forming a second portion of the matrix, wherein the concentration of the at least one therapeutic agent in the matrix is different in the first and second portions of the matrix.

22. The method of Claim 21, wherein the first and second homogenous solutions include a solvent and the first and second homogenous solutions are solidified by evaporation of the solvent.

23. The method of Claim 22, wherein the solvent comprises a miscible organic solvent.

24. The method of Claim 23, wherein the miscible organic solvent is selected from the group consisting of dimethyl sulfoxide, N-methyl pyrrolidone, ethyl lactate, and simple alcohols.

25. The method of Claim 22, wherein the solvent and the polymeric binder are both non-water soluble.

26. The method of Claim 25, wherein the non-water soluble solvent is selected from the group consisting of a poly(lactide-co-glycolide) polymer, N-methyl pyrrolidone, ethyl lactate, anisole, chloroform, tetrahydrofuran, xylene, and methylene chloride.

27. The method of Claim 21, further comprising:
applying successive homogeneous solutions to the matrix; and
solidifying the successive homogeneous solutions, thereby forming additional portions of the matrix, wherein the concentration of the therapeutic agent in the matrix is different in the successive portions of the matrix.

28. The method of Claim 21, wherein applying the first homogeneous solution to the body of the implantable medical device comprises:
introducing the homogeneous solution into a recess in the body of the expandable medical device.

29. The method of Claim 21, wherein applying the first homogeneous solution to the implantable medical device body comprises:
introducing the homogeneous solution into an opening passing through the implantable medical device body.

30. The method of Claim 21, wherein applying the first homogeneous solution to the body of the implantable medical device comprises:
coating a surface of the body of the expandable medical device with the first homogeneous solution.

31. The method of Claim 21, wherein the polymeric binder of the first homogeneous solution is water soluble.

32. The method of claim 21, wherein the therapeutic agent is selected from the group consisting of antithrombotic agents , antiproliferative agents, and antirestenotic agents.

33. The method of Claim 21, wherein the matrix is selected from the group consisting of poly(lactide-co-glycolide) (PLGA) and Poly vinylpyrrolidone (PVP).

34. The method of Claim 21, further comprising:
applying a solution of a barrier material prior to applying the first homogenous solution, the barrier material forming a barrier to the passage of the therapeutic agent in the first homogenous solution to one side of the body of the implantable medical device.

35. The method of Claim 21, wherein the second homogenous solution contains no therapeutic agent.

36. The method of Claim 21, wherein the second homogeneous solution comprises the at least one therapeutic agent, and wherein a concentration of the at least one therapeutic agent in the second homogeneous solution is different from a concentration of the at least one therapeutic agent in the first homogeneous solution.

37. The method of Claim 37, further comprising:
applying successive homogeneous solutions to the matrix; and
solidifying the successive homogeneous solutions, thereby forming additional portions of the matrix, wherein the concentration of the at least one therapeutic agent in the matrix is different in the successive portions of the matrix.

38. A method of forming an implantable medical device configured to release at least one therapeutic agent therefreom, wherein the therapeutic agent is disposed in a matrix affixed to a body of the implantable medical device, and wherein a concentration of the at least one therapeutic agent in the matrix varies as a continuous gradient relative to a surface of the implantable medical device body, the method comprising:
forming a homogeneous solution comprising a polymeric binder and a solvent;
evaporating the solvent in the homogeneous solution, thereby forming a matrix;
exposing the matrix to a solution comprising the therapeutic agent for a time sufficient to produce a partial diffusion of the therapeutic agent into the matrix such that the concentration of the therapeutic agent varies in the matrix; and
affixing the matrix to the implantable medical device body.

39. The method of Claim 39, wherein the matrix is affixed to the implantable medical device body by placing the matrix into the body prior to immersing the matrix in the solution comprising the therapeutic agent.

40. The method of Claim 39, wherein the matrix is affixed to the implantable medical device body by placing the matrix into a recess in the implantable medical device body.

41. The method of Claim 39, wherein the matrix is affixed to the implantable medical device body by placing the matrix into an opening passing through the implantable medical device body.

42. The method of Claim 39, wherein the matrix is affixed to the implantable medical device body by disposing the homogeneous solution comprising a polymeric binder and a solvent into an opening and then evaporating the solvent.

43. The method of Claim 39, wherein the matrix is affixed to the implantable medical device body by coating a surface of the implantable medical device with the matrix.

44. A method for treating a patient by local delivery of at least one therapeutic agent, the method comprising:
delivering an implantable medical device into the body of a patient, the implantable medical device having a matrix affixed to a body of the implantable medical device with concentration of the at least one therapeutic agent in the matrix varying as a gradient relative to a surface of the body matrix with a high concentration of the therapeutic agent spaced a distance from the matrix surface and a low concentration of the therapeutic agent at the matrix surface; and
delivering the therapeutic agent at a release rate and over an administration period determined by the gradient of therapeutic agent in the matrix.
